(19) United States
(12) Patent Application Publication
(10) Pub. No.: US 2022/0250060 A1
(43) Pub. Date: Aug. 11, 2022

(54) INTEGRATED, POINT OF SALE, BLOOD TESTING SYSTEMS AND METHODS

(71) Applicant: Philip E. Werthman, Los Angeles, CA (US)

(72) Inventor: Philip E. Werthman, Los Angeles, CA (US)

(21) Appl. No.: 17/622,705

(22) PCT Filed: Jun. 24, 2020
(86) PCT No.: PCT/US2020/039465

§ 371 Date: Dec. 23, 2021

Related U.S. Application Data
(60) Provisional application No. 62/865,326, filed on Jun. 24, 2019.

(51) Int. Cl. B01L 3/00; G01N 1/34; G01N 27/414

(52) U.S. Cl. CPC B01L 3/502715; B01L 3/502753; G01N 1/34; G01N 27/4145; B01L 2400/0487; B01L 2200/04; B01L 2300/0636; B01L 2300/0819; G01N 27/4146

(57) ABSTRACT

An integrated system for automatically analyzing in real time an analyte in a sample containing fluid is disclosed. The system includes a fluid separator for receiving the sample and separating therefrom a fluid component that contains the analyte, a non-optical, chemical analyte sensing device having at least one sensor for chemically analyzing the analyte, and a microfluidic channel for transferring at least a portion of the fluid component from the separator to the sensing device. In a preferred embodiment, the system is point of care, single-use cartridge in a base unit that separates plasma from a small sample of whole blood and tests an analyte of interest in the plasma.

### RELATED APPLICATIONS

This application claims the benefit of U.S. Provisional Application No. 62/865,326 filed Jun. 24, 2019.

### TECHNICAL FIELD

The present invention relates generally to the field of fluid testing and in particular to systems and methods for preparing and analyzing said fluid, such as blood plasma, in an integrated, small form factor package.

### BACKGROUND

Fluid, or liquid, testing is a common activity for biologic, organic and other substances for the purpose of analyzing constituents of the fluid. Often the liquid material in its pre-existing or natural state needs to be processed first in a separation step in order to extract the fluid of interest (analyte) to be tested.

Human and animal fluid testing, and in particular blood testing, is one of the most common, safe and important diagnostics tools available for checking the health of humans and other living beings. Blood tests are often the first thing doctors and labs administer for checking patient health and for identifying certain diseases and conditions. These tests can help evaluate the health of organs, diagnose diseases such as cancer, HIV/AIDs, diabetes, anemia, coronary heart disease. They can check whether a person has risk factors for heart disease, help determine if medicines are working, and assess how well blood is clotting. It is thus well understood that patient blood is drawn and tested at routine checkup's, acute situations and everything in between.

In the conventional blood test, a sample of “whole blood” is typically taken by a nurse or lab worker by drawing from a vein in an arm using a needle or a finger prick and into a test tube. The test tube is then taken to a lab for analysis of the blood. If the test is to count the blood cells (red or white) or platelets, then the whole blood itself is used. But most other testing is of analytes (the substance whose chemical constituents are to be identified and measured) contained in the fluid that carries the blood cells, called plasma or serum. Indeed, human blood plasma may be the most important and one of the most convenient sources of circulating biomarkers. Plasma contains an abundance of proteins many of which can be used as biomarkers, indicating the presence of certain diseases in an individual, from cancer to Alzheimer's to sepsis. More specifically, blood plasma is mostly water (up to 95% by volume), and contains many proteins (e.g., serum albumins, globulins, and fibrinogen), glucose, clotting factors, electrolytes, hormones, carbon dioxide and oxygen. It plays a vital role in an intravascular osmotic effect that keeps electrolyte concentration balanced and protects the body from infection and other blood disorders.

In order to obtain plasma for testing the many analytes, the whole blood sample must be separated into its component parts in a process called “blood fractionation.” This separation can done by a number of different known processes, but the most common one is called “centrifugation”—typically spinning a tube of fresh whole blood extracted from the patient (often containing an anticoagulant) in a centrifuge that spins the sample until the blood cells fall to the bottom of the tube. The yellowish blood plasma is then poured or drawn off, and then moves on to the analysis step, usually conducted by a trained lab technician. It is thus understood that since in many or perhaps most instances, patient blood is not drawn at a facility (or in the same office) that has its own blood lab, e.g., it is drawn at a doctor's office, the tube containing the patient's whole blood must be transported to a lab, whether a third-party lab at another physical location or, if in a hospital setting, to the hospital's in-house lab, often on different floor or building than where the blood is drawn. There, the tube of blood is loaded into a conventional (tabletop or other sized) centrifuge machine, often with tubes of others' blood, and separated. Then, a trained technician removes some plasma from the tube, runs the tests on the analytes in the plasma that is requested by the doctor, and records the results.

Unfortunately, this conventional, ubiquitous, three-step, often three-location process of (a) whole blood collection; (b) whole blood fractionation, or more generally, blood processing; and (c) analyses of desired analytes in the plasma is inefficient. For one, much more blood than actually needed for testing a range of analytes is collected. The fractured process is understandably costly. And, as much of the public can attest, the process from blood draw to test results in the hands of the caregiver and the patient is very slow. This disjointed multi-location process means that blood test results often take days or even weeks to come back to the “point-of-care” (POC) caregiver—e.g., the doctor—who ordered the testing and then to the patient (the “multi-day clinical lab cycle” problem). Thus, there remains a need for effective, alternative processes and systems that are capable of more efficiently and rapidly detecting a range of analytes from blood than is presently available, i.e., solving the multi-day clinical lab cycle problem. While this problem is well-recognized, to be sure, what some might call the “holy grail” solution—effectively processing and analyzing a small plasma blood sample (1) accurately, (2) safely, (3) rapidly, (4) inexpensively, (5) automatically (i.e., without the need for a blood lab technician), (5) without the need for reagents or fluorescent or other labeling of the blood samples; and all done (6) at the “point-of-care” of the patient—has been elusive.

Indeed, much work is being done to address various aspects of this blood testing, and more generally, biologic fluid testing, inefficiency problem. One area offering promise takes advantage of advances in the field of microfluidics. For example, U.S. Pat. No. 8,221,701 to Cho, et al., titled “Centrifugal force-based microfluidic device for blood chemistry analysis” describes a microfluidics device that can automatically perform various types of blood chemistry analysis. The system relies on centrifugal forces to separate the blood into various chambers to be diluted and mixed with reagents. U.S. Pat. No. 8,663,583 to Kelly, et al., describes a disposable blood analysis cartridge adapted to be used at the point-of-care of a patient, such as in a doctor's office, in the home, or elsewhere in the field. The system includes a sample collection reservoir with an absorbance measurement channel and an optical light scattering channel where a negative or positive pressure is used to push or pull the fluid between the reservoirs, channels and an optical measuring device. This system, however, is limited to optical measurement of blood samples. Unfortunately, optical analysis methods have a number of drawbacks, especially for home or POC applications. Such systems are large, fragile, and expensive. They also need to be calibrated, the specimens must be diluted and/or amplified due to limited sensitivity, and they are not amenable to multiplexing.

U.S. Pat. No. 10,156,579 to Gibbons, et al. titled, “Methods for the detection of analytes in small-volume blood samples”, was an attempt at a full solution to problem. This patent purportedly disclosed a method and system capable of detecting multiple analytes in a small volume of blood samples using microfluidic systems. This method contemplates biofluid transfer from a portion of the device that prepares or separates whole blood by a process such as centrifugation, and delivers the processed fluid to a system that allows the blood to react with reagents to yield a colored product whose wavelength can be detected by an optical reader or other optical spectro-photometrical device. This system delivers the specimen onto a semiconductor chip with a bioassay layer that claimed to obviate the need for any type of specimen amplification, and would chemically react with the specimen to produce light of a specific wavelength for measurement with optical detection device, and a reader that would read out the results. While the system of this invention attempted to combine the steps of whole blood fractionation and the diagnostics of desired analytes, unfortunately, the invention required labeling, optics or reactants of the samples and used optical measurement techniques, providing less than ideal performance.

Others are working on improved methods and systems for separating plasma from whole blood beyond conventional centrifuges. Some have managed to miniaturize the centrifuge into a small handheld device, such that small samples of whole blood can injected into a chamber in a disposable cartridge and powered by an external motor in a base. Other innovations include the plasma separation membranes, such as those from Pall Corporation, capable of generating plasma from whole blood samples.

While each of these innovations and others address one or the other shortcoming of conventional blood plasma testing, none have adequately addressed the multi-step blood testing problem with a self-contained, accurate solution that robustly and cost-effectively works as intended.

Accordingly, what is needed are integrated, cost-effective, automated point-of-care solutions in compact packages that integrate the whole blood fractionation process with blood plasma diagnostics that produces in real time or near real-time the results of testing of multiple analytes in the plasma without using optical or other spectro-photometrical technologies and the associated needs for reagents.

More broadly, what is also needed is an automated solution in a compact and disposable package that combines liquid separation technology for isolating liquid samples with analytics technology for testing analytes of interest in the samples and for obtaining desired test results right on the spot.

### SUMMARY

The present invention meets these needs by disclosing an automated solution in a compact, preferably portable and disposable, fluid testing system, package or unit. that comprises (a) liquid separation technology for isolating an analyte of interest, (b) micro-fluidically-connected to (c) a non-optical, chemical analyte sensing device, that receives the analyte via microfluidic action and that analyzes said analyte to obtain one or more desired test results in real time or near real-time. In preferred embodiments of the analyte sensing device can be a micro-sensing “lab on a chip” or a simple lateral flow immunoassay strip detector.

The present invention meets these needs for specifically blood plasma isolation and testing by disclosing a preferably real time, point-of-care (POC), biologic fluid separation and testing system and method. The system comprises an integrated, self-contained, fluid testing unit or cartridge and a base reader unit that connects to, and preferably receives a preferably disposable cartridge. The cartridge receives a biologic fluid, such as a whole blood sample, and processes it, such as by separating and obtaining plasma from the whole blood sample, and analyzes it in real time. When the analysis generates results electrically, the cartridge may be connected to an electrically-powered base reader unit, which in turn, records the results of the analysis and preferably reads out the results on a display. Thus, in a preferred implementation, the inventive fluid test cartridge can be easily inserted, popped or snapped into the base reader and popped out and disposed of when the test results are read and recorded, with the base reader unit ready to accepts a new cartridge containing a new sample, all at the POC.

In one preferred embodiment, the inventive cartridge contains a disc centrifuge or other fluid processing device to separate the fluid as needed in order to prepare it for testing. It further has a port that receives the processed fluid for transmission through a microfluidics system, which automatically passively or actively causes the fluid to flow therethrough and onto a analyte sensing device, such as a semiconductor bioassay microprocessor “lab on a chip” or a lateral flow immunoassay strip system.

In the embodiment where the chemical sensing system is a biochip, this chip can preferably directly measure specific and multiple analytes or other target molecules and transmits the results to the reader unit. In one preferred embodiment, the base or reader unit or module removably attaches to the cartridge and contains a motor to drive a centrifuge in the disposable cartridge that separates the biologic fluid, e.g., whole blood, into the component needed for testing, and may also generate a pressure head to move the separated fluid within the system. Alternatively, the fluid may passively move through the system via capillary action. In preferred embodiments, the base reader unit may contain a circuit board, LED display screen, wireless module to transfer data and an internal and/or external power supply.

In further detail, the disclosed invention provides a means and method to analyze very small volumes of blood in the order of 30 microliters per test well.

In further detail, the disclosed invention provides a means and method to actively or passively control the motion of the fluid within the cartridge system.

In further detail, the disclosed invention provides a means and method to transfer any necessary reagents from a single or multiple wells onto the microchip biosensor layer via a microfluidics system.

In further detail, the disposable self-contained cartridge fits securely into a base module that contains a motor to drive the centrifuge disc within the cartridge and generate centrifugal force, and may generate negative or positive pressure sufficient to cause the fluid to move through the system onto the surface of the chip to contact the wells containing the testing reagents.

In further detail, capillary action may be the force that draws the fluid from the centrifuge onto the microchip-testing surface.

In further detail, the disclosed invention provides a means and method for the disposable cartridge microprocessor chip to contact a circuit board in the base module to allow transfer of the signal from the chip onto a reader in the base module.

In further detail, the disclosed invention provides a means and method for the base module to visually display results.

In further detail, the disclosed invention provides a means and method for the base module to wirelessly transmit data resulting from testing performed on the microprocessor to a physician or laboratory electronic medical record.

In further detail, the disclosed invention provides a means and method for the centrifuge disc to be housed in the disposable cartridge.

In further detail, the disclosed invention provides a means and method for the microfluidics system to attach to the centrifuge disc at one end and the microprocessor chip at the other.

In further detail, the disclosed invention provides a means and method to control the timing, sequence, and motion of the fluid within the cartridge system in accordance with control laws embedded in an electronic control unit.

In further detail, the disclosed invention provides a means and method to control the timing and motion of the fluid within the cartridge system with one or more valves in the fluidics system.

It is to be understood that the present invention is not limited in its application to the details of construction and the arrangement of components described hereinafter and illustrated in the drawings and photographs. Those skilled in the art will recognize that various modifications can be made without departing from the scope of the invention.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a diagrammatic side view of one embodiment of a fluid separation and analysis system in a cartridge according to the present invention:

FIG. 2 is a diagrammatic top view of one embodiment of a microfluidic “lab on a chip” system (MLOC) used by at least one embodiment of the present invention;

FIG. 3 is a diagrammatic side view of one specific embodiment of the system in a cartridge of FIG. 1, wherein the fluid separator comprises a centrifugation system.

FIG. 4 is diagrammatic side view of one embodiment of the system of the present invention comprising the cartridge shown in FIG. 3 connected to a base unit;

FIG. 5 is a top view of the system shown in FIG. 4; and

FIG. 6 is flow diagram showing one method of the present invention as implemented with the system of FIGS. 4 and 5.

### DETAILED DESCRIPTION OF THE PREFERRED EMBODIMENTS

Referring now to the drawings, like reference numerals designate identical or corresponding features throughout the several views. The present invention discloses an integrated system for analyzing in real time an analyte in a sample containing liquid. It should be understood that the present invention can be implemented for analyzing analytes in biologic fluids (such as blood or urine) or non-biological fluids that require a fluid separation stage as a precursor to chemically analyzing the separated fluid of interest.

The system comprises a fluid separator 210 for receiving the sample and separating therefrom a fluid component that contains the analyte; a non-optical, chemical analyte sensing device 50 having at least one sensor for chemically analyzing the analyte; and a microfluidic channel 180 fluidly connecting the separator to the non-optical, chemical analyte sensing device for transferring at least a portion of the fluid component from the separator to the sensing device. Combining and interconnecting these processes—i.e. fluid separation, transmission via the one or more microfluidic channels to the analyte sensing device, and analysis by the sensing device—into one integrated package is what enable the sample to be analyzed in real time. As used throughout, “real time” means the actual time during which all of these processes in the integrated system occurs. This is to be understood in contrast with conventional blood processing that does not occur in not real time, where the places and times of blood collection, separation and plasma analysis may all be different. steps of blood separation In practice, “real time” could be mere minutes or even seconds.

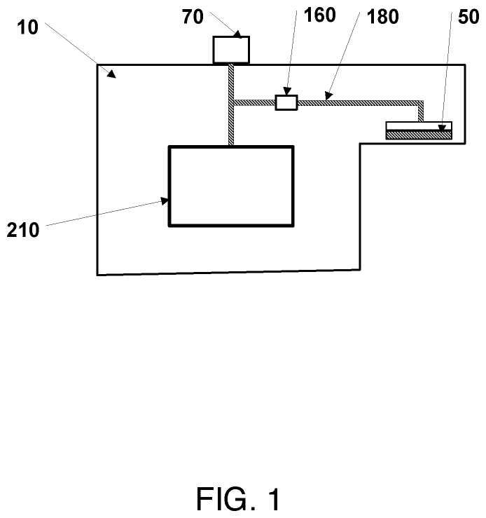

FIG. 1 shows diagrammatically a side view of the components of one embodiment of an all-in-one, automated separator/analyzer of the present invention in the form of a cartridge. As seen, the cartridge 10, has a sample inlet port 70 connected to a liquid separator 210 for directly loading (inserting or injecting) therein a relatively small amount of the sample. The cartridge includes a chemical analyte sensing device 50 that is fluidly connected to an output valve 160 via the microfluidic channel system 180. In the preferred embodiment of the system, the components are integrated together in a single-use, disposable self-contained cartridge, having first processed the sample in separator 210 and then analyzed the separated fluid on a single use chemical analyte sensing device 50.

The invention will now be described as implemented for one preferred system embodiment, namely a system for processing a patient's whole blood for the analysis of multiple components in the patient's blood plasma. In such a system, the present invention discloses an integrated, automated system 10 for analyzing in real time an analyte in the plasma of a sample of whole blood. This system comprises a blood separator 210 for receiving the whole blood sample and separating blood plasma therefrom; a microfluidic channel 180 fluidly connected to the separator for transmitting at least a portion of the plasma from the separator; and a non-optical, chemical plasma analyte sensing device 50 that receives and analyzes plasma from the microfluidic channel. The whole blood sample may comprise less than 1 milliliter of whole blood and preferably between 20 microliters and 1 milliliter of whole blood. The microfluidic channel may actively or passively transmit the portion of the plasma to the sensing device.

A relatively new technological advance that holds great promise to revolutionize the field of biological fluid or “biofluidics” analysis and diagnostics, and that enables one embodiment of the present invention, is the development of label-free, semiconductor biosensor microchips integrated with microfluidics devices for the analysis of fluid samples. These small form factor labs-on-a-chip (LOC) can offer low cost, fast, label-free, highly sensitive yet not fragile, sensing and chemical analysis of analytes in small samples of fluids such as blood plasma. Using LOC's enable small and portable form factors, such as lightweight tabletop systems and even battery powered systems. These new biosensor microchips comprise multiple highly sensitive biosensor transistors—such as those disclosed in U.S. Pat. No. 9,645,135, titled “Nanowire field-effect transistor biosensor with improved sensitivity”—designed on a very small semiconductor chip, or microchip. These new generation of sensors can now (a) directly detect with good sensitivity and scalability and quantify any number of biological molecules (analytes) deposited on their surfaces; (b) be multiplexed,—meaning multiple biosensors can reside on a single chip, with each sensor capable of being prepared with a different reagent to test for a different chemical constituent, all done simultaneously, and (c) convert these results into electrical signals (data) for further processing and readout.

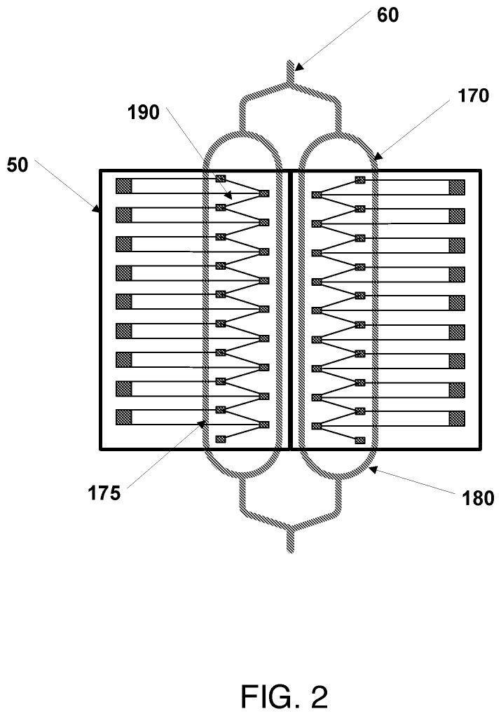

The inventor of the present invention has recognized that such a biosensing LOC device can be designed in a microfluidic system as the non-optical, chemical analyte sensing device of the present invention. Thus, coupling such a device to a small form factor fluid separation system 210, such as a mini-centrifugal or membrane-type blood plasma extraction system, via one or more microfluidic channels 180, all packaged in an integrated unit, like a sterile cartridge, creates a portable, low cost, disposable, truly point-of-care, plasma-separating and analyte detection system that can truly revolutionize and disrupt the entire blood testing industry. FIG. 2 shows one exemplary implementation of a non-optical, chemical analyte sensing device 50 shown in FIG. 1, namely, a microfluidic biosensing “lab on a chip” (MLOC) device 50. As diagrammatically shown, fluid sample containing analytes of interest (biologic or otherwise) is drawn into a microfluidic system 180 at inlet 60 and through microfluidic channels 170, 175 of microfluidic system 180. This fluid transport channels are overlaid on a multiplexed lab on a biosensing microchip LOC 50, such as one of the biosensing chips designed by Selfa, Inc., a portfolio company of the California NanoSystems Institute (CNSI). This flow causes small drops of the sample flowing through the channels to be deposited on sensing zones, or “wells,” 190 of highly sensitive, label-free, multiwire nanowire field effect transistor (mwFET's) biosensors disposed on the chip surface, with each well 190 being prepared with a reactant specific for a measurement of interest. Each well 190 can thus be prepared to analyze a different analyte, simultaneously (i.e., multiplexed). These wells chemically react with the biomolecules that are deposited thereon in order to analyze them for the specific analyte being tested for. The reactions in each well in turn generates electrical signals on the microchip indicative of the analyte reaction in that well, hence providing electronically recordable and readable test results. Thus, in the blood plasma testing use case described herein, plasma fluid travels through channels 170 and 175, depositing along the way plasma fluid on all the wells 190, each prepared with a reactant designed for a specific blood test.

In one preferred embodiment of the non-optical, chemical analyte sensing device 50, a semiconductor LOC chip 50 comprises a surface that contains a biological layer with multiple wells containing antibodies or oligonucleotides or any other molecule used to test specific analytes or other target molecules that may be loaded into the wells.

The present inventive system thus combines into a single package any suitable non-optical chemical analyte sensing device, such as a LOC described herein, with any suitable blood separation technology that can be fluidly connected to the analyte sensing device and packaged therewith in a relatively compact and preferably disposable package, or cartridge. While the following embodiments show this aspect of the invention in the form of a cartridge, it should be understood however, that the form of device is not essential to the present invention, and persons of ordinary skill in the art can readily select a suitable form for a given application. Thus, while the term “cartridge” will be used hereinafter, it should be understood to mean any such suitable form for this combined microfluidic separator/analyzer package. Further, the microfluidic cartridge of the present invention may be constructed from any suitable material, such as a sterile, transparent plastic, mylar or latex, using any method such as injection molding or lamination, and it may be made as a disposable package for one-time use, or otherwise.

Turning now to the liquid separator, any known separator technology that effectively in real time separates fluid containing an analyte of interest from a sample may be used. In plasma use case, the whole blood separator built into the cartridge of the present invention may be any of the new test-tubeless blood separation technologies that can in real time and in small form factor separate plasma from whole blood sample. Non-limiting examples include centrifuge technologies, such as the plasma centrifugation technology designed by Sandstone Diagnostics, any plasma separator member device (e.g, from Pall Corporation or from Spot On Sciences, Inc.), microfluidic filter systems that draw whole blood through the filter using any known drawing method (such as with piezoelectric pumps, micro-syringe pumps, electroosmotic pumps, and the like, or those driven by inherently available internal forces as gravity, hydrostatic pressure, capillary force, absorption by porous material or chemically induced pressures or vacuum, including the microfluidic systems described in U.S. Pat. No. 7,419,638 to Micronics, Inc.), or any other plasma separating and collecting device that can be suitably designed with a micro-fluidic technology to supply the plasma to the biosensing LOC.

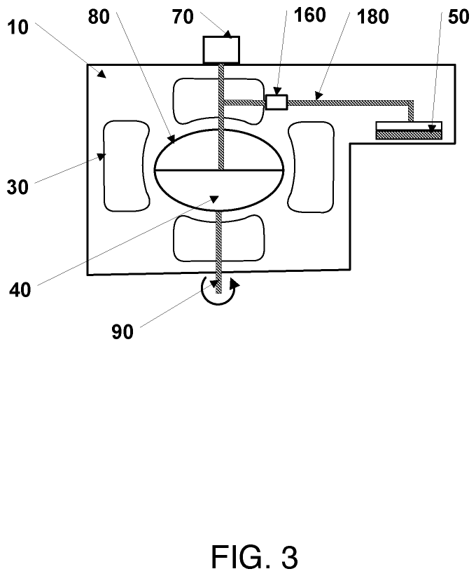

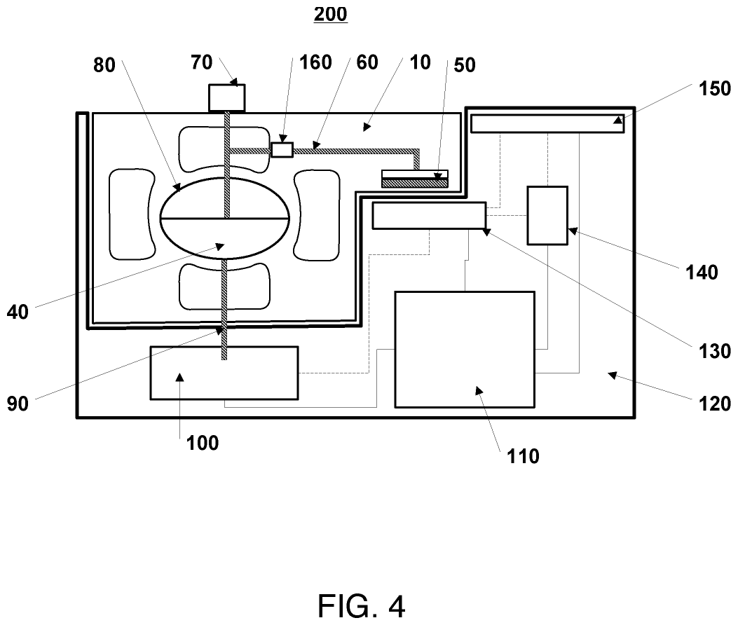

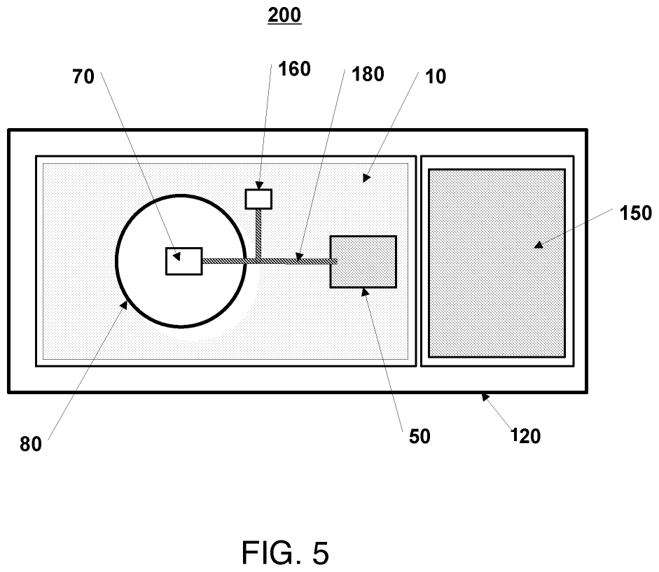

Accordingly, FIG. 3 shows a side view of a specific implementation of the disposable cartridge 10 shown in FIG. 1, with the blood separator 210 implemented as a miniaturized centrifuge 80 (and its components 30, 40 and 90) that engages a motor connectable to the cartridge, such as the centrifuge designed by Sandstone Diagnostics. This is explained in further detail in connection with FIGS. 4 and 5.

FIG. 4 shows a side view and FIG. 5 shows a top view of a real time, analyte diagnostic Point of Care (POC) system 200 of the present invention, comprising the disposable cartridge 10 shown in FIG. 3, as physically placed in and mechanically and electrically connected to an electrically-powered base unit 120. As seen in all three figures, the cartridge 10 has a blood access port 70 connected to the small centrifuge 80 having a centrifuge disc 40 for directly loading (inserting or injecting) therein (with no test tube) a relatively small amount of a patient's whole blood. As seen in FIGS. 3 and 4, four (4) guides 30 hold the disc 40 in place when rotating at high speed. The underside of disc 40 is connected to a fixed, rotatable rod 90, which, when engaged in base unit 20 (FIG. 4), engages motor 100 for translating the rotation of the motor when activated to the centrifuge 80. As described above, cartridge 10 includes a non-optical, chemical analyte sensing device 50 such as biosensor microprocessor chip, LOC 50, that is fluidly connected to plasma output valve 160 via the microfluidic channel system 180. In the preferred embodiment, the cartridge 10 is a single-use, disposable self-contained cartridge, having processed the patient's blood on a single use centrifuge 80 and then analyzed the plasma on the single-use biosensor microprocessor chip 50.

In one preferred embodiment of the real time, analyte diagnostic Point of Care (POC) system 200, the base unit 120 contains a power source (not shown), a motor 100, an electronic control unit 110, a circuit board 130, a visual display 150 for displays test results, and, preferably, a wireless communications module 140. Alternatively, or additionally, the unit 20 may include storage (not shown) for digitally storing results of testing. It will be understood that base 20 can be powered by any suitable power source (e.g, AC or battery) and its electronics can comprise any conventional electronics components that can be designed and programmed as needed in a small form factor (e.g., portable or table-top) to achieve the desired actions (e.g., programmably driving the motor 100 via unit 110) and the desired results (e.g., designing the circuit board 130 to process the signals from LOC 50, programming the controller 110 to receive the analyte data from board 130 and drive the display 150 to displaying test results).

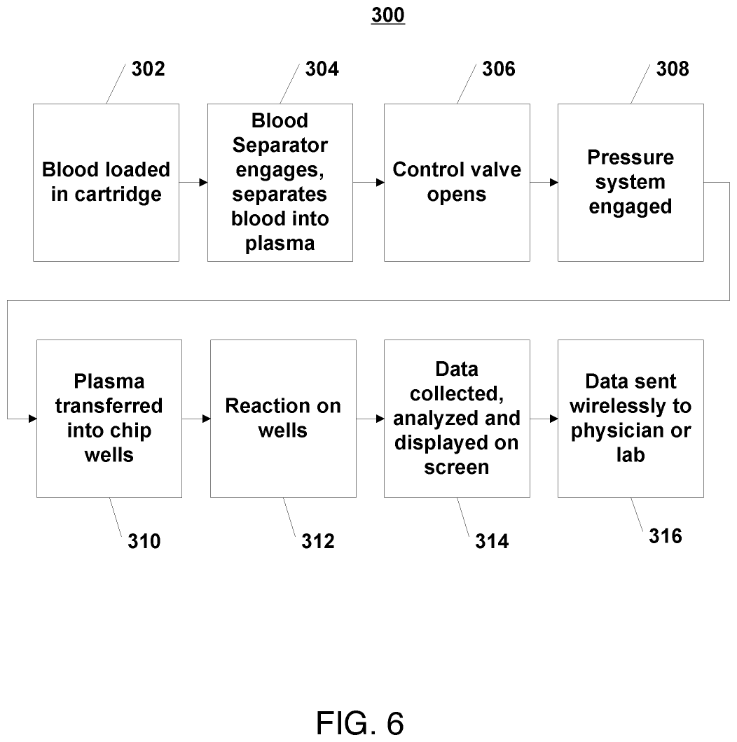

Flow diagram 300 in FIG. 6 shows the operation of the POC testing system of the present invention according to the embodiments shown in FIGS. 2-5. In step 302, a small amount of whole blood is loaded into the cartridge 10, and specifically into the blood separator 210 (or 80) via inlet port 70. From this point forward, the process is fully automated and is completely self-contained and thus sterile. Upon powering on the POC system, blood separator, in step 304, engages the sample to automatically separate out the blood cells, leaving the plasma to be processed. In the case of the centrifuge, when the cartridge 10 secured to the base 20, and is loaded with whole blood, the base 20 may be turned on (automatically or manually) and engaged via the electronic control unit 110. The motor 100 then spins the centrifuge 80 rapidly for a prescribed or programmed period of time (e.g. for less than 90 seconds) via rod 90, separating the blood so that the plasma is extractable. In this embodiment, in step 306, the electronic control unit 110 then opens the valve 160 on the microfluidics transfer channel 180, and activates, in step 308 the motor 100 to produce negative pressure through the tubing system 180 that extends over analyte testing device LOC 50. Thus, in step 310 plasma that was drawn through the fluid transfer channel 180 bathes the biosensing wells 190 on the chip 50 (FIG. 2). In step 312, a biochemical reaction occurs on each of the wells 190. This is where the “magic” happens, whereby in a preferred embodiment that uses multiplexed biosensors, the sensors of each well simultaneously test the analytes desired for, and chip 50 convert the results into electric signals that are sent to the circuit board 130 on base unit 20 for processing. In step 314, the circuit board 130 is programmed to collect and compile the signals as results data which is then—driven by controller 110—visually displayed on the screen 150. The data may optionally be stored in storage, and/or sent out in step 316 to remote storage or to directly a physician wireless device or lab via wireless communications module 140.

While embodiments of the invention have been illustrated and described, it is not intended that these embodiments illustrate and describe all possible forms of the invention. Various changes, modifications, and alterations in the teachings of the present invention may be contemplated by those skilled in the art without departing from the intended spirit and scope thereof. It is intended that the present invention encompass such changes and modifications.

What is claimed is:

1. An integrated, automated system for analyzing in real time an analyte in the plasma of a sample of whole blood, comprising:
a. a blood separator for receiving the whole blood sample and separating blood plasma therefrom;
b. a microfluidic channel fluidly connected to the separator for transmitting at least a portion of the plasma from the separator; and
c. a non-optical, chemical analyte sensing device that receives and analyzes plasma from the microfluidic channel.

2. The system of claim 1, wherein the whole blood sample comprises less than 1 milliliter of whole blood.

3. The system of claim 1, wherein the sample comprises between 20 microliters and 1 milliliter of whole blood.

4. The system of claim 1, wherein the microfluidic channel actively transmits the portion of the plasma.

5. The system of claim 1, wherein the microfluidic channel passively transmits the portion of plasma.

6. The automated system of claim 1, wherein the chemical analyte sensing device is a biosensor microchip that generates an electrical signal from a bio-chemical reaction in the plasma.

7. An integrated system for analyzing in real time an analyte in a sample containing fluid, comprising:
a. a fluid separator for receiving the sample and separating therefrom a fluid component that contains the analyte;
b. a non-optical, chemical analyte sensing device having at least one sensor for chemically analyzing the analyte; and
c. a microfluidic channel fluidly connecting the separator to the non-optical, chemical analyte sensing device for transferring at least a portion of the fluid component from the separator to the sensing device.

8. An apparatus for testing in real time an analyte in a sample of whole blood, comprising:
a. a blood separator for receiving the sample and separating therefrom blood plasma;
b. a biosensor microchip having at least one biosensor for detecting and analyzing, label-free, at least one analyte in the plasma; and
c. a microfluidic subsystem fluidly connecting the separator and the microchip, the subsystem having a channel for transmitting a portion of the plasma from the separator onto the biosensor.

9. The apparatus of claim 8, wherein the biosensor comprises a nanowire field effect transistor (nwFET).

10. The apparatus of claim 8, wherein the microchip contains a plurality of biosensors.

11. The apparatus of claim 10, wherein the microchip is multiplexed such that each biosensor is adapted to detect a different analyte in the plasma.

12. The apparatus of claim 10, wherein the microchip is adapted to detect multiple analytes in the plasma simultaneously.

13. The apparatus of claim 8, wherein the separator, microchip and microfluidic subsystem are contained in a cartridge.

14. The apparatus of claim 8, wherein the cartridge is a point-of-care (POC), single-use and disposable cartridge.

15. The apparatus of claim 8, further including a reagent processing well connected to the microfluidic subsystem.

16. The apparatus of claim 8 wherein the blood separator is a centrifuge.

17. The apparatus of claim 13, further includes an electronic base unit connected to the cartridge.

18. The apparatus of claim 17, wherein the cartridge is removably connected to the electronic base unit.

19. The apparatus of claim 17, wherein the base unit comprises
a. a power source;
b. a circuit board electronically connected to the microchip for receiving electronic signals from the microchip representative of the detected analyte as results data;
c. a display screen for displaying results of the testing; and
d. a control unit for controlling the results data from the circuit board and the display screen.

20. The apparatus of claim 18, further including an electric motor removably connected to the blood separator in the cartridge for driving the separator.

21. The apparatus of claim 18, wherein the electric motor is connected to a pressure subsystem that creates in the microfluidic subsystem negative or positive pressure to compel movement of plasma across the microfluidic channel and toward the microchip.

22. The apparatus of claim 19, further including a wireless module to transfer results data external to the base unit.

23. The apparatus of claim 19, wherein the control unit electronically controls the sequence and timing of movement of fluid from the separator area to the microchip.

24. The apparatus of claim 19, wherein the control unit electronically controls the timing and movement of reagents through the microfluidic system.

25. The apparatus of claim 17, wherein the base unit is connected to a second cartridge as claimed in claim 6, the base unit capable of processing and reading samples obtained from both cartridges containing different blood samples.

26. A single use cartridge for conducting a plurality of immunologic and DNA/RNA/protein testing assays in real-time on charged molecules, ions, and/or chemicals in a whole blood sample obtained from a subject, the cartridge comprising:
a. a whole blood sample processing device for processing the sample;
b. a semiconductor, label-free assay microprocessor detection chip containing biosensor detection wells capable of detecting analytes, oligos or other molecules in the processed blood sample;
c. a receiving cavity configured to receive the blood sample and provide the sample to the processing device; and
d. a microfluidic system that transmits at least a portion of the processed sample from the processing apparatus to the microprocessor detection wells.

27. The cartridge of claim 26, wherein the sample comprises less than 1 milliliter of whole blood.

28. The cartridge of claim 26, wherein the sample comprises between 20 microliters and 1 milliliter of whole blood.

29. A method for processing in real time whole blood in a self-contained cartridge, the method comprising:
a. depositing a sample of whole blood into a blood separator in the cartridge;
b. separating the sample in constituent parts to isolate plasma in the sample;
c. drawing, via microfluid transmission, a portion of the plasma toward a bio-sensing microchip in the cartridge;
d. detecting an analyte in plasma deposited on a biosensor disposed on the microchip; and
e. transmitting an electrical signal representative of the detected analyte to a processor to be recorded and/or displayed as digital data.

30. A system for testing a sample for an analyte of interest, comprising:
a. a separator for receiving the sample and separating therefrom a fluid for testing;
b. a microchip having at least one sensor for chemically detecting, label-free, at least one analyte in the fluid; and
c. a microfluidic subsystem fluidly connecting the separator and the microchip, the subsystem having a channel for transmitting a portion of the fluid from the separator on a sensor on the microchip.
